(19) United States
(12) Patent Application Publication
(10) Pub. No.: US 2015/0201863 A1
(43) Pub. Date: Jul. 23, 2015

(54) METHOD AND APPARATUS FOR DETERMINATION OF A SUITABLE TABLE POSITION FOR AN MR EXAMINATION STEP

(71) Applicant: Siemens Aktiengesellschaft, Muenchen (DE)

(72) Inventors: Aaron Flammang, Tustin, CA (US); Bernd Hengelein, Neunkirchen a. Br. (DE); Christof Krellmann, Erlangen (DE)

(73) Assignee: Siemens Aktiengesellschaft, Muenchen (DE)

(21) Appl. No.: 14/603,726

(22) Filed: Jan. 23, 2015

(30) Foreign Application Priority Data
Jan. 23, 2014 (DE) 102014201242.2

(51) Int. Cl. A61B 5/055; G01R 33/54; G01R 33/30

(52) U.S. Cl. CPC A61B 5/0555; G01R 33/307; G01R 33/543

(57) ABSTRACT

In a method and apparatus to determine a suitable table position for an examination step in a magnetic resonance (MR) apparatus, the patient table is moved to a first table position and a first measurement volume is selected. Adjustment measurements to adjust components of the MR apparatus to the first measurement volume are implemented and the measurement results are stored. A second measurement volume is selected. The difference between an optimal table position for the second examination step and the first table position is calculated. MR data are acquired from the second measurement volume at the optimal table position for the second examination step if the difference exceeds a defined threshold, and at the first table position with the first adjustment parameters if the difference does not exceed the threshold.

### BACKGROUND OF THE INVENTION

1. Field of the Invention

The invention concerns a method to determine a suitable table position for an examination step in a magnetic resonance tomography apparatus (MR apparatus), as well as a corresponding MR apparatus and a data storage medium encoded with programming instructions to execute such a method.

2. Description of the Prior Art

Magnetic resonance tomography (MRT) is used often in everyday clinical environments. The basic requirement for an MRT examination is an optimally homogeneous basic magnetic field, which is normally provided by the basic field magnet of an MR apparatus. The homogeneity is normally sufficiently high in a region around the isocenter of the basic field magnet of the MR apparatus. However, the farther from the isocenter, the lower the homogeneity, which can lead to distorted MR images. The other components of an MR apparatus, such as the gradient system and the RF coils, are also optimized only for measurements within a defined examination region.

For an MR examination, the subject to be examined (normally a person or patient) is placed on a movable patient table which is then driven into the examination region of the MR apparatus. The technician executing the examination then selects an examination volume and an examination protocol and executes an examination step, for example the acquisition of one or more slice groups from which appropriate slice images are reconstructed. If an additional examination volume with a different position should then be selected, there is the question of whether the patient table should be shifted for this second examination so that the second examination volume is also situated as close as possible to the isocenter, or within the homogeneous region of the MR apparatus. Such a selection of the always-optimal table position leads to good image quality, but after every displacement of the patient table, certain adjustment measurements must be executed again, for example the procedure known as shimming, in which possible distortions of the basic magnetic field due to the presence of the subject to be examined (susceptibility artifacts, for example) are compensated as much as possible by the adaptation of the current flow by compensation coils (known as shim coils). This takes time and thereby lengthens the measurement.

It is known to establish the selection of the table position according to different modes, and the technician must select a mode before the measurement. According to a first mode, the optimal table position is calculated and used for each examination step. Every change of the position of the volume produces a recalculation and modification of the table position. In other modes, the technician can define the table position by a manual input of a value. However, the selection of these different modes is an additional work step for the technician.

### SUMMARY OF THE INVENTION

An object of the invention is to provide a method to determine a suitable table position for an examination step in an MR apparatus, that allows an optimally time-saving examination, is simple to apply and delivers good examination results, but without requiring an explicit selection of a mode (from the aforementioned modes, for example).

This object is achieved via the method according to claim 1, the MR apparatus according to claim 11 and the digital data medium according to claim 13.

The method according to the invention is applicable to all types of MR apparatuses, both for medical and non-medical applications. In the latter case, the movable patient table is a subject mount. In most embodiments, the patient table is can be moved or driven by suitable motors, wherein the movement is controlled by a control unit of the MR apparatus. The patient table can be driven in at least one direction, which in typical medical MR apparatuses is usually the axial direction (z-direction). However, the patient table can also be movable in one or two of the other spatial directions, for example it can be adjustable in the height direction.

As used herein, an examination step is the data acquisition of a defined measurement volume. The measurement volume can include one or more slice groups from which MR data are simultaneously acquired. As used herein, a slice group means multiple slices that are parallel to one another. An examination step can be in any examination or data acquisition that can be implemented with an MR apparatus, for example the acquisition of spectroscopy data of a defined measurement volume as well as the acquisition of a three-dimensional image data set of a measurement volume.

The selection of the individual measurement volumes can take place in localizer images obtained advance within the scope of an overview measurement, or in images that have already been acquired.

According to an embodiment of the invention, the patient table is initially driven into a first table position and there a first measurement volume is selected from which MR data should be acquired in a first examination step. This can also occur in a reverse order: first, the measurement volume is selected and then the patient table is driven to the corresponding table position. The first table position is preferably chosen so that the first measurement volume is situated in the homogeneous region; for example, the first table position is the optimal table position for the first measurement volume. In this context, “optimal” means only that a specific suitable table position is calculated or established for a defined measurement volume within the scope of the measurement precision of the specific MR apparatus. Naturally this is not the ideally or “perfectly” optimal table position, but rather is optimal insofar as it can be determined by the MR apparatus. For example, the calculation of the optimal table position can include the calculation of the focal point of the measurement volume, wherein the optimal table position is then chosen so that the focal point is situated as close as possible to the isocenter of the MR apparatus.

In a next step, adjustment measurements are then implemented for adjustment of components of the MR apparatus with regard to the first examination step, and these measurement results are stored as first adjustment parameters. In this step, for example, shimming can be implemented in which the currents in the shim coils are adjusted so that a maximum homogeneity of the basic magnetic field is established in the first examination volume. These settings of the shim coils are then stored.

Typically, MR data are then acquired from the first measurement volume in a first examination step, preferably with these adjustment parameters. However, this acquisition step is not necessary for implementation of the method according to the invention since this merely accesses the stored adjustment parameters. However, the adjustment measurements are normally automatically added before an examination step as necessary, meaning that normally adjustment measurements and examination step are executed in direct succession. The adjustment parameters are preferably stored together with the respective table position and/or the position and size of the respective measurement volume.

A second measurement volume from which MR data should be acquired in a second examination step is then selected (normally by the user). For example, an additional slice group can be positioned at a different location in the body of the patient. The MR apparatus thereupon preferably calculates an optimal table position for the second examination step, wherein here as well “optimal” is to be understood in the sense explained above. According to one embodiment of the invention, this occurs via calculation of the optimally exact mathematical middle point of the planned measurement volume. If the patient table can only be moved in one direction, this middle point is preferably projected on the axis along which the patient table can be moved. The position along this axis is then the optimal table position. If the patient table can be moved along multiple spatial directions, the table position in which the middle point of the measurement volume is optimally close to the isocenter, or to the middle point of the homogeneous region of the MR apparatus is accordingly assumed as the optimal table position.

A difference is thereupon calculated between the calculated optimal table position and the first table position, or between the focal points of the first and second measurement volume. If the patient table can only be moved in one direction, the difference is preferably a scalar quantity which indicates the deviation of the two table positions along this direction as a measure of length In other embodiments, the difference can also be a vector quantity, in particular if the difference between the focal points is calculated. The focal point is preferably the mathematical middle point of the measurement volumes, but can also be calculated in a different manner; for example, the respective tilt of the measurement volume can be considered. The focal point can be used for the calculation of the optimal table position, but other calculation methods are also possible; in particular, instead of the mathematical middle point a point can be used that is also affected by other factors.

The method furthermore accesses a threshold, wherein this threshold can be generated in different ways, be input by the user or be stored in the MR apparatus. The threshold can depend on different factors that are explained in detail further below.

The calculated difference from the threshold is thereupon compared. If the difference exceeds the threshold, the patient table is preferably driven to the calculated optimal table position (insofar as it is not already located there) in order to acquire MR data from the second measurement volume there. Adjustment measurements to adjust components of the MR apparatus with regard to the second examination step or with regard to the second measurement volume are preferably implemented beforehand, and the measurement results are stored as second adjustment parameters. If the difference falls below the threshold, the already known first table position is selected. Insofar as the patient table is not still located there, it is driven to this first table position and the first adjustment parameters are used in order to acquire MR data from the second measurement volume.

Therefore, the method has the advantage that the patient table does not need to be moved for every new examination step. Rather, an acceptable deviation of the position from (for example) a mathematically calculated optimal position is introduced via the threshold. If the difference is only slight, no repositioning of the patient table is necessary, which has as a consequence a significant time savings. For example, the patient table often does not need to be moved once, and most of all it is not necessary to implement the cited adjustment measurements again. An optimization of the entire examination duration can thereby be achieved in that table positions are selected that have already been used in previous measurement steps, and thus results from previous measurements can be adopted.

The invention also has the advantage that the position determination no longer takes place rigidly by the selection of one of the fixed configuration settings of the MR apparatus, which are then identical for all system and application variations with regard to the determination of the position. Rather, the position determination takes place flexibly and can be adapted to the requirements of the specific examination, or even to every individual examination step.

The method can also be used for additional examination steps. For example, a third measurement volume is selected and an optimal table position for this third measurement value is calculated again. The difference is then preferably calculated not only at the first table position but also for every additional table position for which adjustment parameters are stored. All differences are then compared with the threshold. If only one difference falls below the threshold, the patient table can be driven to the table position of this already known measurement volume. The adjustment parameters belonging to this measurement volume or, respectively, to the already implemented examination step are thereupon resorted to, and this is used for the acquisition of the MR data from the third measurement volume.

In the following, various possibilities are described for determination of the threshold:

According to a first embodiment, the threshold is adjustable by a user, either via manual input or via selection from multiple predetermined values. This can occur once before implementation of the examination so that the same threshold applies to all examination steps. Alternatively, it can be readjusted for every examination step.

According to an alternative embodiment, the threshold is provided depending on the type of examination step. In particular, the threshold can be predetermined depending on: the RF coils used for the examination steps; the size of the measurement volume; the body part to be measured; the type of examination; and/or the physiological state of the patient to be measured. This has the advantage that the requirements of the different examinations with regard to the position volume can be considered. For example, there are types of examinations which do not depend on precise positioning, for example because distortions at the edge of the field of view are not so severe. Here a higher threshold can then be selected, this a high tolerance for the table position. In contrast to this, in other examinations—for example in a scientific context—the table position should be as good as possible, such that the threshold is chosen to be rather low. Furthermore, the RF coil that is used—the local coil, for example—also determines the threshold. Given only a locally active RF coil (a hand coil, for example), the threshold is accordingly low. Multiple RF coils can also be used which possibly have different respective thresholds.

The threshold can thus be adapted system-specifically and application-specifically, either for the individual measurement step or in advance in the form of a preset. For the invention, only the adaptation or selection of an individual tolerance parameter (threshold) is preferably still required.

According to a preferred embodiment, the user (technician) can select a category for the threshold before the examination. For example, the category could designate the type of examination (for example “Science” for high precision) or the question posed for the examination (for example “Heart” or “Skull”). For each category, either a specific threshold or multiple thresholds are stored that in turn depend on one or more of: the RF coils used for the examination steps; the size of the measurement volume; the body part to be measured; and the physiological state of the patient to be measured.

According to a further embodiment, the threshold of the difference can be of different sizes in different spatial directions. This in particular concerns the embodiment in which the threshold is specified as a vector, for example because it is calculated as a difference between the focal points of the measurement volumes. This has the advantage that here again the configuration of the MR apparatus (including the RF coils used) can be taken into account. The magnetic field is often more homogeneous in one direction than in other spatial directions.

The adjustment measurements are preferably executed automatically by the MR apparatus and, for example, include the following adjustments: on the one hand, the shim currents can be set; the reception dynamic of the analog/digital converter (ADC) can also be adapted. The frequency of the RF system can also be set to the resonance frequency of the basic magnetic field and/or the transmission power of the RF pulses (transmitter adaptation). The adjustment measurements can include one or more of the cited adjustments. The shim is preferably implemented as a 3D shim in which the shim volume is limited to the respective measurement volume (local shim).

The invention also concerns a corresponding MR apparatus configured to implement the method described above. The MR apparatus preferably has a control unit that is configured to control the MR apparatus so that the method according to the invention is executed. In particular, the control unit can be a rule-based controller. It is preferably integrated into a control console of the MR apparatus.

The invention also concerns data storage medium encoded programming instructions with (program code) that cause an MR apparatus to execute the method according to the invention when the program code is executed in the control unit of an MR apparatus.

### BRIEF DESCRIPTION OF THE DRAWINGS

FIG. 1 schematically depicts an embodiment of an MR apparatus according to the invention.

FIG. 2 is a schematic sectional view of an embodiment of an MR apparatus according to the invention.

FIG. 3 is a flow chart of an embodiment of the method according to the invention.

FIG. 4 is a schematic plan view of an examination region in a second examination step.

FIG. 5 is a schematic plan view of the examination region of FIG. 4 in a third examination step.

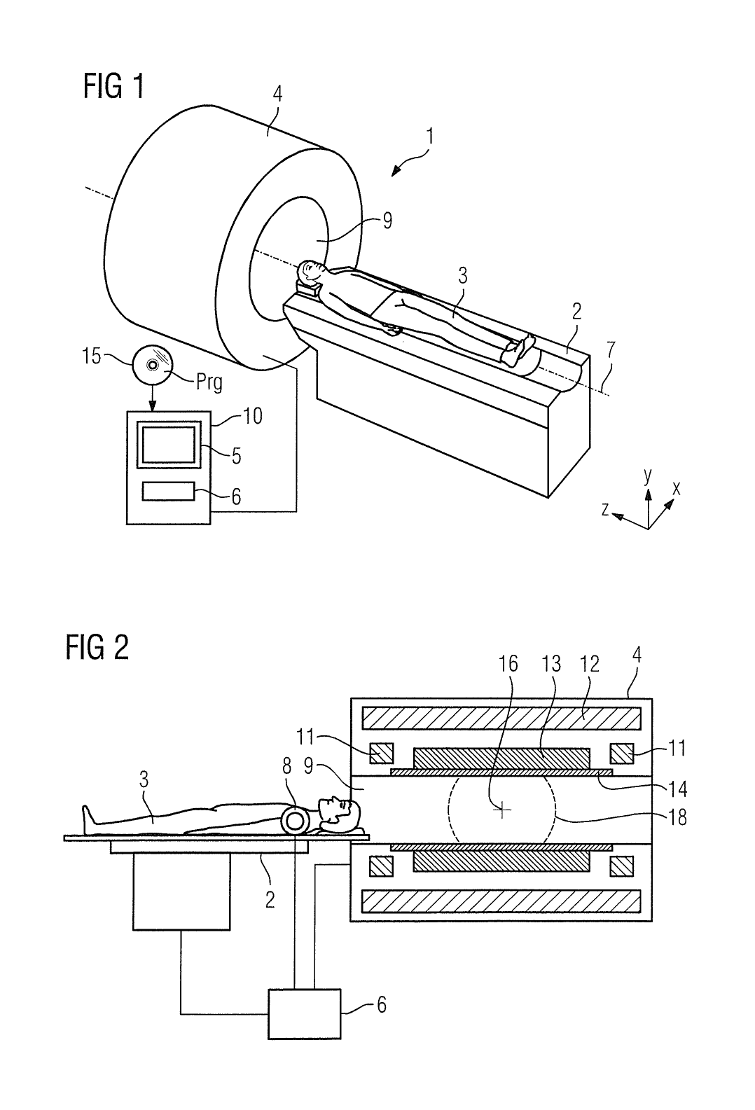

### DESCRIPTION OF THE PREFERRED EMBODIMENTS

According to the shown embodiment, an MR apparatus 1 designed according to the invention has a basic field magnet 4 with an examination region 9 situated in the inner space (bore) of the basic field magnet 4. A patient 3 is laid on a patient table 2 that can be driven into the examination region along the apparatus axis 7. This occurs with suitable motors (not shown). In the presented example, the patient table 2 can be moved only along the axis 7, i.e. along the z-direction. However, an adjustment capability in the x- and y-directions is also possible.

The shown MR apparatus is controlled by the control unit 6, which is typically part of a control console 10. The control unit 6 is typically a computer or part of a computer, for example the central processing unit (a CPU). Memory modules (for example a hard disk or RAM or other data storage) for storing predetermined thresholds can also be part of the control unit 6. The control console 10 has a monitor 5 as well as input units such as a keyboard and mouse (not shown), which allow a user to enter a threshold. A program that includes program code segments Prg to implement the method according to the invention can be stored on a digital storage medium 15 (for example a digital, optical or magnetic data storage, for example a CD-ROM) and thus can be uploaded into the control unit 6.

FIG. 2 again shows in longitudinal section the patient table 2 which can be moved in the axial direction 7, with the patient 3 laid thereupon. A local RF coil 8 is arranged on the shoulder of the patient.

In the longitudinal section through the primary magnet 4, the components located therein are visible: the basic magnetic field is generated by the coil 12, which normally is comprised of superconducting material. A gradient system 13 is also present, which generates the gradient fields. Corresponding shim coils 11 are likewise schematically depicted. An RF body coil is provided with the reference character 14. The presentation in FIG. 2 is purely schematic, and the spatial arrangement of these components can be different than as depicted.

A strong magnetic field which is sufficiently homogeneous (in particular within the region 18) for the acquisition of MR data (in particular image data) is generated by the basic field magnet 4. In many apparatuses, the homogeneous region is centered around the isocenter 16.

The MR apparatus 4 (including the components included therein) as well as the patient table 2 and the RF coil 8 are again controlled by the control unit 6. Additional components of the MR apparatus—for example ADCs, frequency generators, amplifiers, filters and other transducers, that may also participate in the adjustment measurements, are not shown in FIG. 2.

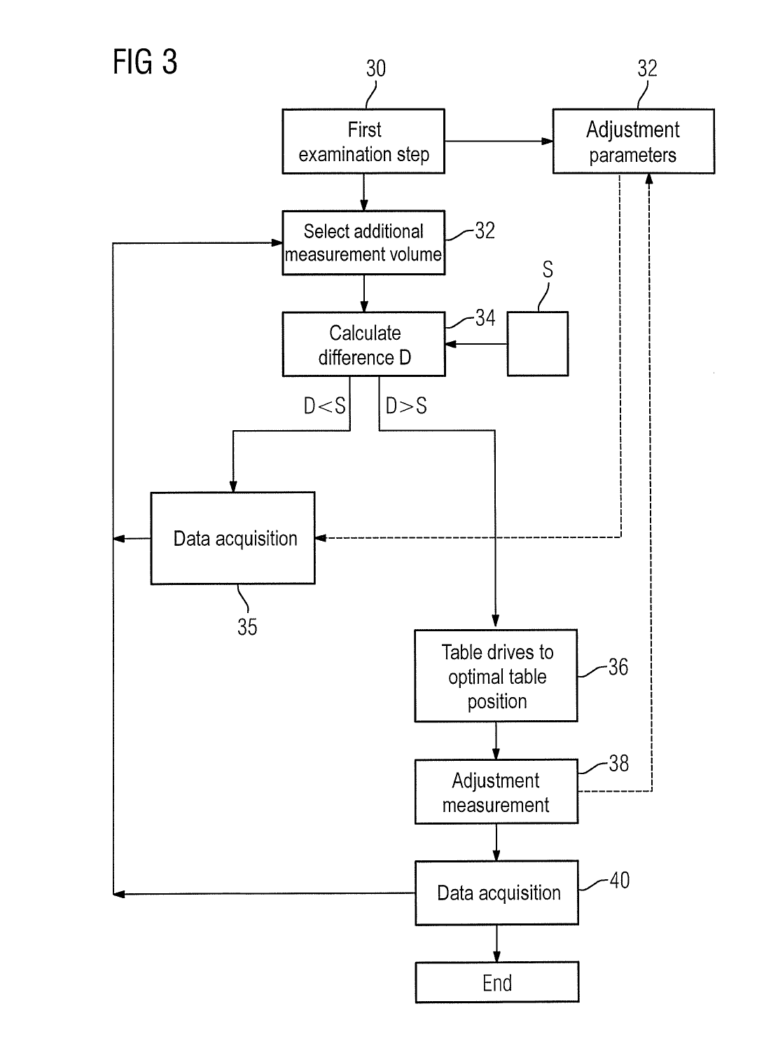

An exemplary embodiment of the method according to the invention is shown in FIG. 3. This begins with a first, defining examination step 30. A first measurement volume is hereby selected, corresponding adjustment measurements are implemented and the adjustment parameters 32 that belong to this measurement volume or, respectively, to this examination step are stored.

An additional measurement volume is thereupon positioned in step 32. For this measurement volume, either its mathematical focal point is calculated or the table position that is optimal for this focal point is already calculated. A difference D—for example between this optimal table position and the table position in the first examination step—is thereupon calculated (step 34). A threshold S is accessed that is either input by the user or is predetermined due to a selected configuration or system setting. If the difference D is smaller than the threshold, the data acquisition 35 is implemented at the first table position, wherein the first adjustment parameters are also used. The repositioning of the measurement volume thus does not produce a variation of the table position for the data acquisition.

However, if the difference is greater than the threshold, the data acquisition is executed at the position that produces the best possible image quality. This means that, in step 36, the table is driven to the optimal table positioning. Corresponding adjustment measurements 38 are implemented and the adjustment parameters 32 are stored for possible later adjustment parameters. The data acquisition 40 thereupon takes place. If no additional examination step is required, the method ends here. Otherwise, it returns to step 32 and an additional measurement volume is selected. The method is then inasmuch repeated, wherein then the second already known tables position is also taken into account; two differences D are thus calculated and the respective smaller of the two is used for comparison with the threshold.

All measurements are executed at an identical table position insofar as they have a difference from one another of the table position that is within the threshold. Exceeding the threshold produces a redetermination of the table position.

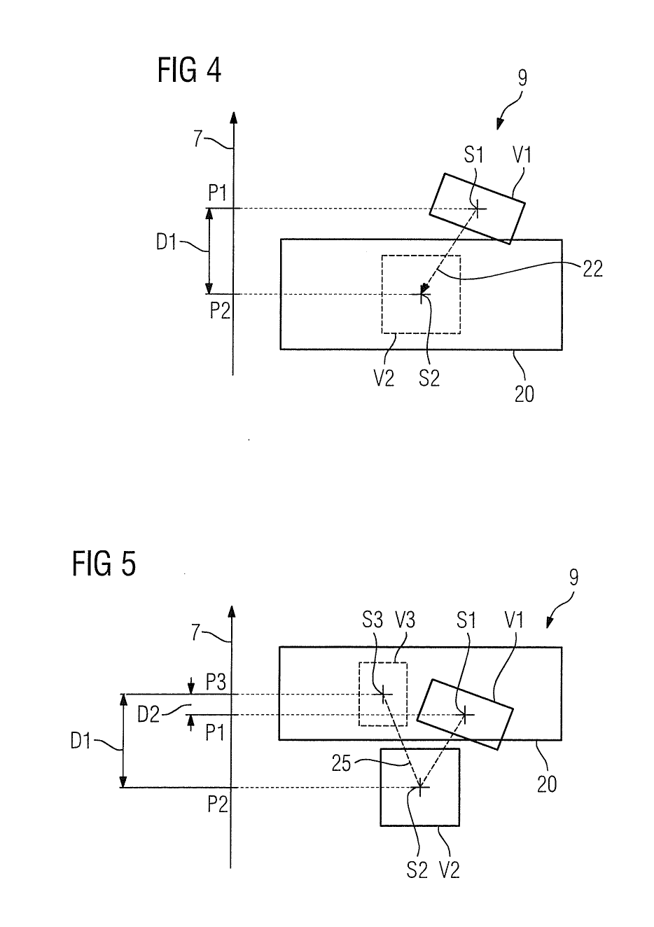

This is shown again in FIGS. 4 and 5. FIG. 4 schematically shows a plan view of an examination region 9 with a first measurement volume V1 that is positioned therein. This first measurement volume V1 has a mathematical focal point S1. Since the corresponding patient table can only be displaced along the axial direction 7, for the calculation of the difference this focal point S1 is projected orthogonally on the axis and the optimal table position P1 thus results at which MR data form the first measurement volume V1 have also possibly been acquired. A new measurement volume V2 is thereupon positioned. According to one embodiment of the method according to the invention, a threshold that is different in different spatial directions is then determined, which here is shown in the form of a tolerance volume 20 which is situated around the second measurement volume M2. Since the volume V1 with the focal point S1 is situated outside of the tolerance volume 20, the optimal table position is recalculated. This again yields the table position P2 via projection on the axis 7. The difference D1 between P2 and P1 exceeds the threshold established in this direction. The threshold could likewise also be established for the difference vector 22 between the focal points S1 and S2. The patient table is therefore moved to the new position P2 where MR data of the measurement volume V2 are then acquired.

FIG. 5 shows a next examination step which should be implemented at a measurement volume V3 with focal point S3. A tolerance volume 20 around the third measurement volume V3 is determined again, as well as an optimal table position Presentation parameters and/or evaluation parameters for this measurement volume. The thresholds can be the same as for the second examination step; they can also have been adapted. Since the focal point S1 of the measurement volume V1 is situated within the tolerance volume 20 for the new measurement volume V3, a recalculation of the table position is not implemented. Expressed in a different way: if the difference D2 between the optimal table positions for the first and third measurement volume is smaller than the provided threshold, the patient table therefore drives to the same position that was used for the acquisition of MR data of the measurement volume V1. The corresponding adjustment parameter for this measurement that has already taken place is then accessed, and the MR examination is implemented at the measurement volume V3 without adjustment measurements taking place again for this purpose.

The described invention has the advantages that, with a single definition of one or more thresholds, the determination of the table positions can be influenced depending on the current examination context for the individual examination step.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to determine a table position for an examination step in a magnetic resonance (MR) apparatus comprising a movable patient table, on which an examination subject is situated, said method comprising:
(a) moving said patient table, with said examination subject situated thereon, to a first table position within said MR apparatus, and selecting a first measurement volume from which MR data are to be acquired from the examination subject in a first examination step of an MR data acquisition procedure, said first measurement volume having a first measurement volume focal point;
(b) conducting adjustment measurements to adjust components of said MR apparatus to parameters that are needed for said first examination step, and storing results of said adjustment measurement in an electronic memory as first adjustment parameters;
(c) selecting a second measurement volume within the examination subject, from which MR data are to be acquired in a second examination step, said second measurement volume having a second measurement volume focal point;
(d) in a computer, calculating a difference selected from the group consisting of a difference between an optimal table position of said patient table for said second examination step and said first table position, and a difference between said first measurement volume focal point and said second measurement volume focal point;
(e) in said computer, comparing said difference to a threshold to obtain a comparison result designating whether said difference does or does not exceed said threshold; and
(f) operating said MR apparatus to acquire said MR data from said second measurement volume in said second examination step, with said patient table at said optimal table position for said second examination step, if said difference exceeds said threshold, and operating said MR apparatus with said first adjustment parameters, and with said patient table at said first table position, to acquire said MR data from said second measurement volume in said second examination step, if said difference does not exceed said threshold.

2. A method as claimed in claim 1 comprising, between (b) and (c), operating said MR apparatus with said first adjustment parameters to acquire MR data from said first measurement volume in said first examination step.

3. A method as claimed in claim 1 comprising, if said difference exceeds said threshold, moving said patient table to said optimal table position for said second examination step and, before acquiring said MR data from said second measurement volume, conducting further adjustment measurements to adjust said components of said MR apparatus for said second examination step and storing measurement results of said further adjustment measurements in said electronic memory as second adjustment parameters, and operating said MR apparatus with said second adjustment parameters to acquire said MR data from said second measurement volume in said second examination step.

4. A method as claimed in claim 1 comprising repeating (c), (d) and (e) for a plurality of repetitions respectively for additional measurement volumes of said examination subject from which MR data are to be acquired respectively in additional examination steps and, in each repetition, calculating said difference from the group consisting of a difference between an optimal table position for an additional examination step and said first table position, and a difference between said first measurement volume focal point and a focal point of the respective additional measurement volume.

5. A method as claimed in claim 1 comprising for each examination step in said MR data acquisition procedure, allowing manual setting of said threshold by manual entry into said computer of a selected value for said threshold, or by manual entry of a selection of said threshold from a plurality of predetermined values of said threshold.

6. A method as claimed in claim 1 comprising, in said computer, automatically setting a value of said threshold dependent on a type of MR data acquisition in the respective examination step.

7. A method as claimed in claim 6 comprising, in said computer, determining said threshold dependent on at least one of a configuration of a radio-frequency coil of said MR apparatus used to acquire the MR data in the respective examination step, a size of measurement volume of the respective examination step, a body part of the examination subject from which said MR data are to be acquired in the respective examination step, a type of said MR data acquisition procedure, and a physiological state of said examination subject.

8. A method as claimed in claim 1 comprising manually selecting a category for said threshold before acquiring said MR data.

9. A method as claimed in claim 8 comprising, in said computer, determining said threshold dependent said category and on at least one of a configuration of a radio-frequency coil of said MR apparatus used to acquire the MR data in the respective examination step, a size of measurement volume of the respective examination step, a body part of the examination subject from which said MR data are to be acquired in the respective examination step, a type of said MR data acquisition procedure, and a physiological state of said examination subject.

10. A method as claimed in claim 1 wherein said patient table is movable in at least one direction, and wherein said difference between said first measurement volume focal point and said second measurement volume focal point is calculated in said direction in which said patient table is movable.

11. A method as claimed in claim 1 wherein said patient table is movable in a plurality of different spatial directions, and comprising selecting said threshold to have a different value dependent on a direction in which said table is movable between said first measurement volume focal point and said second measurement volume focal point.

12. A method as claimed in claim 1 comprising conducting said adjustment measurements from the group consisting of shimming, adapting a reception dynamic of analog-to-digital converter of said MR apparatus used to detect said MR data, adjusting a frequency of a radio-frequency system to a resonance frequency of said MR apparatus for a given basic magnetic field, and adjusting a transmission power of radio-frequency pulses employed in said MR apparatus to acquire said MR data.

13. A magnetic resonance (MR) apparatus comprising:
an MR data acquisition unit comprising a movable examination table on which an examination subject is situated;
a control computer configured to move said patient table, with said examination subject situated thereon, to a first table position within said MR apparatus, and to select a first measurement volume from which MR data are to be acquired from the examination subject in a first examination step of an MR data acquisition procedure, said first measurement volume having a first measurement volume focal point;
said control computer being configured to operate said MR apparatus to conduct adjustment measurements to adjust components of said MR apparatus to parameters that are needed for said first examination step, and to store results of said adjustment measurement in an electronic memory as first adjustment parameters;
said control computer being configured to select a second measurement volume within the examination subject, from which MR data are to be acquired in a second examination step, said second measurement volume having a second measurement volume focal point;
said control computer being configured to calculate a difference selected from the group consisting of a difference between an optimal table position of said patient table for said second examination step and said first table position, and a difference between said first measurement volume focal point and said second measurement volume focal point;
said control computer being configured to compare said difference to a threshold to obtain a comparison result designating whether said difference does or does not exceed said threshold; and
said control computer being configured to operate said MR apparatus to acquire said MR data from said second measurement volume in said second examination step, with said patient table at said optimal table position for said second examination step, if said difference exceeds said threshold, and to operate said MR apparatus with said first adjustment parameters, and with said patient table at said first table position, to acquire said MR data from said second measurement volume in said second examination step, if said difference does not exceed said threshold.

14. A non-transitory, computer-readable data storage medium encoded with programming instructions, said storage medium being loaded into a control computer of a magnetic resonance (MR) apparatus that comprises a movable patient table on which an examination subject is situated, said control instructions causing said control computer to:
move said patient table, with said examination subject situated thereon, to a first table position within said MR apparatus, and select a first measurement volume from which MR data are to be acquired from the examination subject in a first examination step of an MR data acquisition procedure, said first measurement volume having a first measurement volume focal point;
conduct adjustment measurements to adjust components of said MR apparatus to parameters that are needed for said first examination step, and store results of said adjustment measurement in an electronic memory as first adjustment parameters;
select a second measurement volume within the examination subject, from which MR data are to be acquired in a second examination step, said second measurement volume having a second measurement volume focal point;
calculate a difference selected from the group consisting of a difference between an optimal table position of said patient table for said second examination step and said first table position, and a difference between said first measurement volume focal point and said second measurement volume focal point;
compare said difference to a threshold to obtain a comparison result designating whether said difference does or does not exceed said threshold; and
operate said MR apparatus to acquire said MR data from said second measurement volume in said second examination step, with said patient table at said optimal table position for said second examination step, if said difference exceeds said threshold, and operate said MR apparatus with said first adjustment parameters, and with said patient table at said first table position, to acquire said MR data from said second measurement volume in said second examination step if said difference does not exceed said threshold.
